(19) United States
(12) Patent Application Publication
(10) Pub. No.: US 2002/0098156 A1
(43) Pub. Date: Jul. 25, 2002

(54) Prophylaxis paste

(76) Inventor: Lori A. Milliron, New Castle, PA (US)

Correspondence Address: Richard L. Byrne, WEBB ZIESENHEIM LOGSDON ORKIN & HANSON, P.C., 700 Koppers Building, 436 Seventh Avenue, Pittsburgh, PA, 15219-1818, US

(21) Appl. No.: 10/054,739

(22) Filed: Jan. 22, 2002

Related U.S. Application Data
(60) Provisional application No. 60/263,043, filed on Jan. 19, 2001.

(51) Int. Cl. A61K 7/16

(52) U.S. Cl. 424/49

(57) ABSTRACT

A prophylaxis paste in which pumice particles are largely, if not completely, substituted with Roxite particles, or an admixture of Roxite particles with amorphous silicon dioxide particles. Other prophylaxis paste constituents are also present which, in the preferred embodiments of the invention, provide improved characteristics compared with the prophylaxis pastes of the prior art.

### CROSS REFERENCE TO RELATED APPLICATION(S)

This patent application claims priority to United States Provisional Patent Application Serial No. 60/263,043, filed Jan. 19, 2001, which is incorporated herein by reference.

### FIELD OF THE INVENTION

The present invention relates to a novel prophylaxis paste for use by dental hygienists.

### DESCRIPTION OF RELATED ART

In spite of steady innovations in dentistry and dental care products, even the most current prophylaxis pastes are unpleasant for patients and dental hygienists alike. Prophylaxis pastes, often denominated “prophy pastes,” typically contain particulate pumice as a suspended abrasive material. Prophylaxis pastes, according to the prior art, contain various constituents including, but not limited to, pumice, water, flavorings, preservatives, sodium saccharin, colorings, sodium silicates, thickeners, surfactant and humectants. For prophylaxis pastes containing fluoride, a trade accepted amount of fluoride is 1.23% of sodium fluoride. Prophylaxis pastes are applied to the teeth with an air-driven rotary cup and are used to remove dental pellicle, plaque and stains and to polish the teeth. Because of the large particle size of traditional pumice and silica particles used, usually about 30 microns in diameter, there is no true polishing effect in that only a matte satin-like finish is attained. Moreover, the pumice particles create an overwhelming gritty mouth feel, and inevitably cause spattering during use as a result of composition non-homogeneity and centripetal forces on the pumice particles. As a result, such compositions are perceived as messy, unpleasant and unsatisfactory by patients and hygienists alike.

Accordingly, a need remains for a novel prophylaxis paste in which maximum polishing effect and minimized gritty mouth feel are accomplished in an otherwise superior composition.

### SUMMARY OF THE INVENTION

In order to meet this need, the present invention is a prophylaxis paste in which pumice particles are largely, if not completely, substituted with Roxite particles, or an admixture of Roxite particles with amorphous silicon dioxide particles. Other prophylaxis paste constituents are also present which, in the preferred embodiments of the invention, provide improved characteristics of the prophylaxis pastes of the prior art.

### DESCRIPTION OF THE PREFERRED EMBODIMENT(S)

The present invention is a prophylaxis paste in which pumice particles are largely, if not completely, substituted with Roxite particles or an admixture of Roxite particles with amorphous silicon dioxide particles. Certain other constituents and amounts of prophylaxis paste compositions contribute to the preferred embodiments of the invention, but the use of Roxite, or Roxite combined with amorphous silicon dioxide, in the described particle sizes, dramatically reduces the gritty mouth feel and splattering associated with pumice-based pastes. Most importantly, the compositions of the present invention, with the substitution of the Roxite or the Roxite/amorphous silicon dioxide particles for pumice, yield a superior polishing compound when contrasted with pumice-based prophylaxis pastes of the prior art.

Roxite is a rare earth mineral known for its ability to polish glass, such as quality optical lenses. Roxite is a fused zirconium oxide-based polishing powder and may be used in the present invention in particles having an average particle size (diameter) of less than 5 microns and preferably about 1.3 microns (finer than 1400 Microgrit Grade). Roxite may be admixed with amorphous silicon dioxide particles, such as are available under the trade name “Zeodent.” Zeodent 113 is a polishing compound with particle sizes (diameters) ranging between 8-13 microns; Zeodent 165 has particles ranging between 10-16 microns. Amorphous silicon dioxide is a well-known dentifrice additive, however, and dentifrice silicas are available from a number of suppliers worldwide. All of these smaller particles differ from the pumice particles of the prior art not only in their smaller size but in their hardness and other characteristics, rendering the claimed particles as improved for true polishing effect.

The present prophylaxis pastes are formulated based upon an aqueous vehicle containing a humectant, such as a polyol (polyhydric alcohol sugar derivative). Polyols control moisture gain or loss and also provide sweetness to the prophylaxis paste without presenting sugars fermentable by oral bacteria. Typical polyols for inclusion in the present prophylaxis paste formulates include, without limitation, sorbitol, glycerol, maltitol, or mixtures thereof, with the mixture of sorbitol and maltitol being preferred.

Various thickening agents are also added for proper gelling and moisture control of the paste. These thickening agents can be guar gum or other vegetable gums, such as Iota Carrageenan, preferably a cold processed Iota Carrageenan.

Polyols alone do not provide the necessary sweetness to the formula, and sodium saccharin is a common sweetener which is non-cariogenic. Certified food colors may also be added for eye appeal, depending on a further flavoring agent, including, without limitation, FD&C Blue No. 1, D&C Red No. 33, FD&C Red No. 6 Lake, FD&C Green No. 3, FD&C Green No. 5 and Yellow No. 1. Flavoring oils, such as mint flavor, cinnamon, bubble gum, fruit flavors, chocolate, or other flavors or combinations may be added. Menthol may be added to add coolness to flavors and to enhance the mouth feel of the paste.

In addition to the above ingredients, therapeutic agents may also be added to the present prophylaxis pastes. As mentioned above, 1.23% sodium fluoride addition is standard in all but fluoride-free prophylaxis pastes. An antibacterial agent, such as Triclosan, may be added to help to prevent cavities, plaque formation and gingivitis. Triclosan is a bisphenol derivative that has bacteriostatic activity against a wide range of gram-positive and gram-negative bacteria, often used in combination with either sodium fluoride or zinc citrate when incorporated in dentifrice products. Other bacteriostatic agents, such as sodium benzoate, may be added to preserve shelf life.

The above-described ingredients are incorporated in the following amounts. The Roxite particles are included in the present prophylaxis pastes in the amount of about 1.03-7.27% by weight, more preferably 2.25-5.25% by weight, most preferably about 4.37% by weight. When present, the Zeodent 113 may be included in the amount of 20.78-28.69% by weight, preferably 20.78% by weight. When present, the Zeodent 165 may be present in the amount of about 2.91-6.23% by weight, more preferably 4.25-5.25% by weight, most preferably 4.98% by weight. Polyols are present in the amount of about 4-12% by weight each; when sorbitol and maltitol are used in combination preferably the sorbitol is present in the amount of about 4.20-12.46% by weight, most preferably 8.31%, and maltitol is incorporated in the amount of about 4.16-12.46% by weight, preferably about 4.16%. Vegetable gum is added in the amount of about 2.07-6.23% by weight, preferably 2.49%. Triclosan may be included in amounts between about 0.30-0.50% by weight, preferably 0.30% by weight. Sodium saccharide, flavors, colors and other additives are added in amounts known in the art.

The invention is further illustrated according to the following Example.

### EXAMPLE I

To 47.07% by weight sterile water were added 4.16% by weight “Maltisweet” brand maltitol and 8.31% by weight “Sorbobrite” brand sorbitol, with mixing. Roxite particles having an average particle size of 1.3 microns were added to the admixture in the amount of 4.37% by weight, together with 4.98% by weight Zeodent 165 and 20.78% by weight Zeodent 113. Flavoring agent was added in the amount of 2.49% by weight, together with 0.83% by weight sodium saccharin, 1.23% by weight sodium fluoride, 2.99% by weight sodium benzoate, 2.49% by weight carageenan, 0.30% by weight Triclosan and an aliquot of coloring agent (percent by weight negligible). After thorough mixing, the resulting prophylaxis paste was ready for patient use and/or shelf storage under sealed conditions.

Although the invention has been disclosed above with reference to particular materials and amounts, the invention is only to be limited insofar as is set forth in the accompanying claims.

The invention claimed is:

1. A prophylaxis paste for dental hygiene use comprising a carrier suitable for use as a dentifrice in combination with about 1.03-7.27% by weight Roxite particles having an average particle size less than 5 microns.

2. The prophylaxis paste of claim 1, wherein said Roxite particles are present in the amount of about 2.25-5.25% by weight.

3. The prophylaxis paste of claim 2, wherein said Roxite particles are present in the amount of about 4.37% by weight.

4. The prophylaxis paste of claim 3, wherein silicon dioxide particles form an additional constituent.

5. The prophylaxis paste of claim 4, wherein said silicon dioxide particles have an average particle size of between about 8 and about 16 microns.

6. The prophylaxis paste of claim 5, wherein said silicon dioxide particles are amorphous silicon dioxide particles.

7. The prophylaxis paste of claim 6, wherein some of said silicon dioxide particles have an average particle size between about 8 and about 13 microns and wherein some of said silicon dioxide particles have an average particle size between about 10 and about 16 microns.

8. The prophylaxis paste of claim 7, wherein said silicon dioxide particles having an average particle size between about 8 and about 13 microns are present in the amount by weight of about 20.78-28.69%.

9. The prophylaxis paste of claim 8, wherein said silicon dioxide particles having an average particle size between about 8 and about 13 microns are present in the amount by weight of about 20.78%.

10. The prophylaxis paste of claim 9, wherein said silicon dioxide particles having an average particle size between about 10 and about 16 microns are present in the amount by weight of about 2.91-6.23%.

11. The prophylaxis paste of claim 10, wherein said silicon dioxide particles having an average particle size between about 10 and about 16 microns are present in the amount by weight of about 4.25-5.25%.

12. The prophylaxis paste of claim 11, wherein said silicon dioxide particles having an average particle size between about 10 and about 16 microns are present in the amount by weight of about 4.98%.

13. The prophylaxis paste of claim 12, wherein a polyol is present.

14. The prophylaxis paste of claim 13, wherein said polyol is selected from the group consisting of sorbitol, glycerol and maltitol.

15. The prophylaxis paste of claim 14, wherein maltitol and sorbitol are present.

16. The prophylaxis paste of claim 15, wherein maltitol is present in the amount of about 4.16-12.46% by weight.

17. The prophylaxis paste of claim 16, wherein maltitol is present in the amount of about 4.16% by weight.

18. The prophylaxis paste of claim 17, wherein sorbitol is present in the amount of about 4.20-12.46% by weight.

19. The prophylaxis paste of claim 18, wherein sorbitol is present in the amount of about 8.31% by weight.

20. The prophylaxis paste of claim 19, wherein a vegetable gum is present in the amount of about 2.07-6.23% by weight.

21. The prophylaxis paste of claim 20, wherein said vegetable gum is carageenan.

22. The prophylaxis paste of claim 21, wherein the paste contains a therapeutically effective amount of a bacteriostat.

23. The prophylaxis paste of claim 21, wherein the paste contains a therapeutically effective amount of Triclosan.
